(19) United States
(12) Patent Application Publication
(10) Pub. No.: US 2026/0308631 A1
(43) Pub. Date: Oct. 8, 2026

(54) KINETICALLY CONTROLLED METHODS TO PREPARE CONCENTRATED SHELF-STABLE NUCLEIC ACID TRANSFECTION VEHICLES

(71) Applicant: The Johns Hopkins University, Baltimore, MD (US)

(72) Inventors: Hai-Quan MAO, Baltimore, MD (US); Yizong HU, Baltimore, MD (US); Jinghan LIN, Baltimore, MD (US)

(21) Appl. No.: 19/479,125

(22) PCT Filed: May 7, 2024
(86) PCT No.: PCT/US2024/028168

§ 371 Date: Oct. 27, 2025

Related U.S. Application Data
(60) Provisional application No. 63/500,843, filed on May 8, 2023.

(51) Int. Cl. C12N 15/864; C07K 14/005; C12N 5/00; C12N 5/073; C12N 15/867; C12N 15/88

(52) U.S. Cl. CPC C12N 15/8645; C07K 14/005; C12N 5/0062; C12N 5/0603; C12N 15/867; C12N 15/88; C12N 2510/04; C12N 2513/00; C12N 2740/15043; C12N 2740/15051; C12N 2750/14143; C12N 2750/14151; C12N 2800/10; C12N 2830/34

(57) ABSTRACT

Compositions of assembly solutions and stabilization solutions that can achieve kinetic control of the size and composition of polycationic/nucleic acid particles and the optimized process for reproducible and scalable production of concentrated, shelf-stable nanoparticle vehicles for in vitro transfection of cell culture in bioreactors are disclosed.

### CROSS-REFERENCE TO RELATED APPLICATIONS

This application claims the benefit of U.S. Provisional Application No. 63/500,843, filed May 8, 2023, the contents of which are incorporated herein by reference in their entirety.

### TECHNICAL FIELD

The presently disclosed subject matter generally relates to methods for preparing concentrated shelf-stable nucleic acid transfection vehicles.

### BACKGROUND

Gene therapy has become increasingly available in treating various genetic and acquired diseases. Most of these therapies are delivered using viral vectors, such as vectors derived from lentivirus, retroviruses, and adeno-associated virus (AAVs). One of the most common methods to produce these vectors is transient transfection of a HEK293 packaging cell line or derivative thereof with plasmid DNAs (pDNAs) encoding viral accessory proteins and a transfer plasmid that contains the vector backbone. Benchmark transfection reagents include calcium phosphate, lipofectamine, and poly(ethyleneimine) (PEI). In a typical transfection procedure using PEI, for example, pDNA and PEI are separately dissolved in a serum-reduced medium and then mixed to form pDNA/PEI nanoparticles through charge-mediated complex coacervation. Following a predetermined incubation period, the complex particles are added into cell cultures. The complex coacervation with PEI facilitates cell entry, endosomal escape, and nuclear transport of pDNAs, resulting in expression of the viral accessory proteins.

Demands for the production of viral vectors, however, have increased. To meet these increased demands, scalable, and reproducible production methods are essential to ensure consistent delivery of safe and efficacious therapeutic outcomes. The widely adopted method to manually prepare pDNA/PEI particles immediately before transfection suffers, however, from high batch-to-batch variations. Accordingly, the field can benefit from a solution to reduce the experimental variability and achieve maximum transfection efficiency using an off-the-shelf pDNA/PEI particle product with case-of-use features and a sufficiently high DNA concentration to reduce dosage volumes.

To address these needs, methods were developed for generating concentrated, stable, off-the-shelf DNA/polyethylenimine (PEI) particles for reproducible transfection of cells to produce viral vectors. See, for example, International PCT Patent Application Publication No. WO2022177980 for Methods for Preparation of Shelf-Stable Plasmid DNA/Polycation Particles with Defined Sizes for Cell Transfection, to Mao et al., published Aug. 25, 2022, (hereinafter “the '980 application”); and International PCT Patent Application Publication No. WO2022177981 for Composition of Shelf-Stable Plasmid DNA/Polyethyleneimine Particles With Defined Sizes For Viral Vector Production and Methods for Preparation, to Mao et al, published Aug. 25, 2022, (hereinafter “'the 981 application”), each of which is incorporated by reference in its entirety. Although these methods simplify and streamline the transfection process, thereby making it operator-independent and facilitating the scale-up production of viral vectors, they use specialized equipment, e.g., multiple confined impinging jet (CIJ) mixers. Accordingly, there is a need for simple methods for preparing off-the-shelf pDNA/PEI particles using routine laboratory apparatus.

### SUMMARY

In some aspects, the presently disclosed subject matter provides a method for preparing a plurality of stable polycationic/nucleic acid complex nanoparticles, the method comprising: (a) providing one or more nucleic acids in a first buffer in a total nucleic acid concentration ranging from 0.1 to 0.8 mg/mL; (b) providing a polycationic polymer in a second buffer, wherein the first buffer and the second buffer can be the same or different; (c) mixing the polycationic polymer with the nucleic acid and incubating mixture for a period of time to form a plurality of polycationic/nucleic acid complexes; and (d) mixing a third buffer with the plurality of polycationic/nucleic complexes to form a plurality of stable polycationic/nucleic acid nanoparticles having a defined particle size.

In certain aspects, the one or more nucleic acids are selected from an antisense oligonucleotide, cDNA, genomic DNA, guide RNA, plasmid DNA (pDNA), vector DNA, mRNA, miRNA, piRNA, shRNA, and siRNA. In particular aspects, the one or more nucleic acids comprise plasmid DNA or a mixture of different species of plasmid DNA. In more particular aspects, the one or more nucleic acids comprise a mixture of one or more plasmid DNAs, wherein the one or more plasmid DNAs comprise a transfer plasmid encoding a lentiviral vector and plasmid DNAs encoding a gag protein, a pol protein, a rev protein, and an env protein.

In certain aspects, the lentiviral vector comprises a modified left (5′) lentiviral LTR comprising a heterologous promoter, a Psi packaging sequence (Ω+), a cPPT/FLAP, an RRE, a promoter operably linked to a polynucleotide encoding a therapeutic transgene, and a modified SIN (3′) lentiviral LTR. In certain aspects, the env protein comprises a VSV-g envelope glycoprotein.

In certain aspects, the one or more nucleic acids comprise a mixture of one or more plasmid DNAs, wherein the one or more plasmid DNAs comprise, a packaging plasmid, a helper plasmid, and a transfer plasmid encoding an adeno-associated viral (AAV) vector.

In certain aspects, the pDNA in a final suspension of the plurality of stable polycationic/nucleic acid nanoparticles has a concentration in a range of about 50 to about 400 μg/mL.

In certain aspects, the first buffer comprises one or more components selected from a non-buffering salt, a monovalent buffering salt, a bivalent buffering salt, a trivalent buffering salt, a buffering salt having a higher valency, and combinations thereof. In particular aspects, the non-buffering salts are selected from a group comprised of sodium chloride, potassium chloride, and combinations thereof; the buffering salts with varying valency are selected from a group comprised of phosphate, citrate, EDTA, pyrophosphate, ATP, tripolyphosphate, hexametaphosphate, bis-Tris, MES monohydrate, Tris base, HEPES, magnesium, calcium, aluminum, Iron (II), Iron (III), and combination thereof.

In certain aspects, the first buffer has a pH from about 2.0 to about 9.0. In particular aspects, the first buffer has a pH from about 7.0 to about 7.4.

In certain aspects, the second buffer comprises one or more buffering salts selected from a non-buffering salt, a monovalent buffering salt, a bivalent buffering salt, a trivalent buffering salt, a buffering salt having a higher valency, and combinations thereof. In particular aspects, the non-buffering salts are selected from a group comprised of sodium chloride, potassium chloride, and combinations thereof; the buffering salts with varying valency are selected from a group comprised of phosphate, citrate, EDTA, pyrophosphate, ATP, tripolyphosphate, hexametaphosphate, bis-Tris, MES monohydrate, Tris base, HEPES, magnesium, calcium, aluminum, Iron (II), Iron (III), and combination thereof.

In certain aspects, the second buffer has a pH from about 2.0 to about 9.0. In certain aspects, the second buffer comprises an acidic solution.

In certain aspects, the third buffer comprises an acidic pH buffer having a pH from about 2.0 to about 9.0. In particular aspects, the acidic pH buffer comprises HCl. In more particular aspects, the acidic pH buffer comprises between about 0.25 mmol/L to 20 mmol/L of protons.

In certain aspects, the third buffer comprises one or more sugars. In particular aspects, the one or more sugars comprise trehalose. In more particular aspects, the one or more sugars comprise between about 2.5% to about 30% w/w of trehalose.

In certain aspects, the polycationic polymer is selected from polyethylenimine (PEI), chitosan, PAMAM dendrimers, protamine, poly(arginine), poly(lysine), poly(beta-aminoesters), cationic peptides and derivatives thereof. In particular aspects, the polycation comprises PEI. In more particular aspects, the PEI comprises linear PEI or modified linear PEI.

In certain aspects, the method further comprises incubating the plurality of polycationic/nucleic acid complexes for a period of time having a range from about 0.5 minutes to about 90 minutes. In particular aspects, the method comprises incubating the plurality of polycationic/nucleic acid complexes at about room temperature (22±4° C.).

In certain aspects, the defined particle size is between about 100 nm to about 1000 nm. In more certain aspects, the defined particle size is between about 400 nm to about 650 nm.

In certain aspects, the first buffer further comprises a cryoprotectant, the second buffer further comprises a cryoprotectant, the third buffer further comprises a cryoprotectant, or combinations thereof. In particular aspects, the method further comprises lyophilizing or freezing the plurality of polycationic/nucleic acid nanoparticles at about −80° C. to form a plurality of lyophilized or frozen polycationic/nucleic acid nanoparticles. In more particular aspects, the method further comprises storing the plurality of lyophilized or frozen polycationic/nucleic acid nanoparticles for a period of time. In certain aspects, the period of time has a range of up to about four months. In certain aspects, the method further comprises thawing the plurality of lyophilized or frozen polycationic/nucleic acid nanoparticles to form a plurality of thawed polycationic/nucleic acid nanoparticles. In certain aspects, the plurality of thawed polycationic/nucleic acid nanoparticles are stable for a period of time up to about 4 hours. In certain aspects, the method further comprises disposing the plurality of thawed polycationic/nucleic acid nanoparticles in a bioreactor to produce one or more transfected cells.

In other aspects, the presently disclosed subject matter provides a method for preparing a lentiviral vector or adeno-associated viral vector, the method comprising contacting one or more cells with a polycationic/nucleic acid nanoparticle prepared by the method described hereinabove.

In certain aspects, the one or more cells comprise HEK293 cells or a derivative thereof. In particular aspects, the one or more cells comprise a cell selected from HEK293S cells, HEK293T cells, HEK293F cells, HEK293FT cells, HEK293FTM cells, HEK293SG cells, HEK293SGGD cells, HEK293H cells, HEK293E cells, HEK293MSR cells, and HEK293A cells. In more particular aspects, the one or more cells comprise HEK293T cells. In even more particular aspects, the one or more cells comprise HEK293T cells adapted for suspension culture.

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE DRAWINGS

The patent or application file contains at least one drawing executed in color. Copies of this patent or patent application publication with color drawings will be provided by the Office upon request and payment of the necessary fee.

Having thus described the presently disclosed subject matter in general terms, reference will now be made to the accompanying drawings, which are not necessarily drawn to scale, and wherein:

FIG. 1A is a schematic showing methods disclosed in the '980 application and the '981 application for preparing pDNA/PEI particles;

FIG. 1B is a schematic showing the presently disclosed methods for preparing concentrated shelf-stable nucleic acid transfection vehicles;

FIG. 2A, FIG. 2B, and FIG. 2C are representative photographs of an example run at 80 mL scale at DNA concentration of 180 g/mL; FIG. 2B is a photograph of (i) 40-mL acidic PEIpro® solution containing cryoprotectant and (ii) 40 mL DNA cocktail solution (pH=7.0-7.4 solution containing certain concentrations of non-buffering salt, bivalent and trivalent buffering salts and cryoprotectant); FIG. 2C is a photograph showing that immediately following mixing, the solution became turbid suggesting formation of DNA/PEI complexes.

FIG. 3A and FIG. 3B are representative photographs of an example run at 80 mL scale at DNA concentration of 180 μg/mL. FIG. 3A is a plot of Z-average particle diameter by dynamic light scattering (nm) versus time; FIG. 3B is a stabilization buffer containing a certain concentration of acidic pH buffer is added at 10% of total volume;

FIG. 4A and FIG. 4B demonstrate that size growth may be monitored by turbidity (absorbance) eliminating the need for expensive DLS modality (optional). FIG. 4A is a plot of absorbance versus complex DLS z-average (nm). Absorbance at 1000 nm gave nearly a linear correlation to DLS size. The monitoring assay can be defined as absorbance not to exceed a certain threshold before stabilization. FIG. 4B shows photographs of illustrating the turbidity at (i) 5 min of size growth, upon which the complexes were measured at a z-average of 400 nm; (ii) 20 min of size growth, upon which the complexes were measured at a z-average of 510 nm; and (iii) 40 min of size growth, upon which complexes were measured at a z-average of 800 nm;

FIG. 5A, FIG. 5B, and FIG. 5C demonstrate the production and stability of uniform pDNA/PEI complexes upon addition of the third buffer. FIG. 5A is a plot of the intensity fraction (%) versus Z-average particle diameter (nm); FIG. 5B is the Z-average particle size (nm) of pDNA/PEI complexes upon addition of the third buffer at 400, 510 and 800 nm, that were either freshly prepared, freshly prepared and then incubated on benchtop under ambient temperature for 4 h, or freshly prepared and then underwent a freeze-thaw cycle respectively; FIG. 5C is a plot of polydispersity index (PDI) of pDNA/PEI complexes upon addition of the third buffer at 400, 510 and 800 nm, that were either freshly prepared, freshly prepared and then incubated on benchtop under ambient temperature for 4 h, or freshly prepared and then underwent a freeze-thaw cycle, respectively. All data were measured at 180 μg DNA/mL. All parameters were comparable to those observed in methods disclosed previously in WO2022177980 and WO2022177981 using a confined impinging jet (CIJ) mixer and pump-mediated mixing at 50 μg DNA/mL;

FIG. 6 demonstrates benchmarked transfection efficiency. Transfection was done with freeze-thawed samples (at 400, 510, and 800 nm) at the same dosage concentration. Control 400-nm samples used the method disclosed in WO2022177980 and WO2022177981. FG control used high PBS concentration to grow building block nanoparticles to 400 nm within 10 min (FIG. 2 in Hu et al., Nano Lett. (2021)). SG control used low PBS concentration to grow building block nanoparticles to 400 nm within 90 min (FIG. 5 in Nano Lett. (2021)). FG and SG have previously shown optimal and comparable transfection efficiency. Data suggest that stability and bioactivity of the resultant pDNA/PEI complexes are not sensitive to the exact timing for adding the stabilization buffer;

FIG. 7A, FIG. 7B, and FIG. 7C show: (FIG. 7A) Size growth of pDNA/PEI complexes mediated by different concentrations of PBS at 200 μg/mL. (FIG. 7B) Size growth of pDNA/PEI complexes mediated by 1×PBS at 200 μg/mL. Left, monitor of Z-average size from mixing (t=0 min) to 30 min; right, monitor of polydispersity index from mixing (t=0 min) to 30 min. (FIG. 7C) Size growth of pDNA/PEI complexes mediated by citrate ions at 200 μg/mL. Left, monitor of Z-average size from mixing (t=0 min) to 30 min.; right, monitor of polydispersity index from mixing (t=0 min) to 30 min;

FIG. 8A, FIG. 8B, FIG. 8C, and FIG. 8D show: (FIG. 8A) Transfection on HEK293T cells with DNA/PEI particles at different sizes containing 10% luciferase plasmid (n=3). Luminescence was measured 48 h after transfection. (FIG. 8B) Transfection on HEK293F cells with DNA/PEI particles at different sizes containing 10% luciferase plasmid (n=3). Luminescence was measured 48 h after transfection. (FIG. 8C) Transfection on HEK293T cells with DNA/PEI particles at different sizes containing 100% tdTomato plasmid (n=3). Left, tdTomato positive cell percentage; right, median fluorescence intensity in tdTomato positive population. Fluorescence was measured 48 h after transfection. (FIG. 8D) Transfection on HEK293F cells with DNA/PEI particles at different sizes containing 100% tdTomato plasmid (n=3). Left, tdTomato positive cell percentage; right, median fluorescence intensity in tdTomato positive population. Fluorescence was measured at 48 h after transfection.

FIG. 9A, FIG. 9B, FIG. 9C, and FIG. 9D show: (FIG. 9A) Mixing flow rates with their according theoretical characteristic mixing time. (FIG. 9B) Z-average size measurements from dynamic light scattering (DLS) of DNA/PEI particles mixed via different methods or flow rates. Particles were assembled in the same buffer and stabilized at the same time point (n=3). (FIG. 9C) Polydispersity index (PDI) reports from dynamic light scattering (DLS) of DNA/PEI particles mixed via different methods or flow rates (n=3). (FIG. 9D) Transfection on HEK293T cells with DNA/PEI particles stabilized at the same defined sizes but mixed via different methods or flow rates (n=3). Luminescence was measured at 48 h after transfection.

### DETAILED DESCRIPTION

The presently disclosed subject matter now will be described more fully hereinafter with reference to the accompanying drawings, in which some, but not all embodiments of the presently disclosed subject matter are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated drawings. Therefore, it is to be understood that the presently disclosed subject matter is not to be limited to the specific embodiments disclosed and that modifications and other embodiments are intended to be included within the scope of the appended claims.

In some embodiments, the presently disclosed subject matter provides a method for preparing a plurality of stable polycationic/nucleic acid complex nanoparticles, the method comprising: (a) providing one or more nucleic acids in a first buffer in a total nucleic acid concentration ranging from 0.1 to 0.8 mg/mL; (b) providing a polycationic polymer in a second buffer, wherein the first buffer and the second buffer can be the same or different; (c) mixing the polycationic polymer with the nucleic acid and incubating mixture for a period of time to form a plurality of polycationic/nucleic acid complexes; and (d) mixing a third buffer with the plurality of polycationic/nucleic complexes to form a plurality of stable polycationic/nucleic acid nanoparticles having a defined particle size.

In certain embodiments, the one or more nucleic acids are selected from an antisense oligonucleotide, cDNA, genomic DNA, guide RNA, plasmid DNA (pDNA), vector DNA, mRNA, miRNA, piRNA, shRNA, and siRNA. In particular embodiments, the one or more nucleic acids comprise plasmid DNA or a mixture of different species of plasmid DNA. In more particular embodiments, the one or more nucleic acids comprise a mixture of one or more plasmid DNAs, wherein the one or more plasmid DNAs comprise a transfer plasmid encoding a lentiviral vector and plasmid DNAs encoding a gag protein, a pol protein, a rev protein, and an env protein.

In certain embodiments, the one or more nucleic acids encode a viral vector. In preferred embodiments, the viral vector is a lentiviral vector. Illustrative examples of lentiviral vectors suitable for use in particular embodiments contemplated herein include but are not limited to vectors derived from HIV (human immunodeficiency virus; including HIV type 1, and HIV type 2); visna-maedi virus (VMV) virus; the caprine arthritis-encephalitis virus (CAEV); equine infectious anemia virus (EIAV); feline immunodeficiency virus (FIV); bovine immune deficiency virus (BIV); and simian immunodeficiency virus (SIV).

In particular embodiments, the transfer plasmid encodes a lentiviral vector that comprises a left (5′) lentiviral LTR, a Psi packaging sequence (Ψ+), a central polypurine tract/DNA flap (cPPT/FLAP), a rev response element (RRE), a promoter operably linked to a polynucleotide encoding a therapeutic transgene, and a right (3′) lentiviral LTR. Lentiviral vectors may optionally comprise post-transcriptional regulatory elements including, but not limited to, polyadenylation sequences, insulators, a woodchuck hepatitis virus posttranscriptional regulatory element (WPRE), a hepatitis B virus (HPRE), and the like.

In particular embodiment, a transfer plasmid a lentiviral vector that comprises a modified left (5′) lentiviral LTR comprising a heterologous promoter, a Psi packaging sequence (Ψ+), a central polypurine tract/DNA flap (cPPT/FLAP), a rev response element (RRE), a promoter operably linked to a polynucleotide encoding a therapeutic transgene, and a modified (3′) lentiviral LTR.

In particular embodiments, a transfer plasmid a lentiviral vector that comprises a modified 5′ LTR wherein the U3 region of the 5′ LTR is replaced with a heterologous promoter to drive transcription of the viral genome during production of viral particles. Examples of heterologous promoters which can be used include, for example, viral simian virus 40 (SV40) (e.g., early or late), cytomegalovirus (CMV) (e.g., immediate early), Moloney murine leukemia virus (MoMLV), Rous sarcoma virus (RSV), and herpes simplex virus (HSV) (thymidine kinase) promoters.

In particular embodiments, a transfer plasmid a lentiviral vector that comprises a modified self-inactivating (SIN) 3′ LTR that renders the viral vector replication defective. SIN vectors comprise one or more modifications of the U3 region in the 3′ LTR to prevent viral transcription beyond the first round of viral replication. This is because the right (3′) LTR U3 region is used as a template for the left (5′) LTR U3 region during viral replication and, thus, the viral transcript cannot be made without the U3 enhancer-promoter. In particular embodiments, the 3′ LTR is modified such that the U3 region is deleted and the R and/or U5 region is replaced, for example, with a heterologous or synthetic poly(A) sequence, one or more insulator elements, and/or an inducible promoter.

In particular embodiments, one or more pDNAs encode a transfer plasmid comprising a packageable viral vector genome and one or more of the viral structural/accessory proteins selected from the group consisting of: gag, pol, env, tat, rev, vif, vpr, vpu, vpx, and nef. In preferred embodiments, the viral structural/accessory proteins are selected from the group consisting of: gag, pol, env, tat, and rev. In more preferred embodiments, the viral structural/accessory proteins are selected from the group consisting of: gag, pol, env, and rev or gag, pol, and env.

Viral envelope proteins (env) determine the range of host cells which can ultimately be infected and transformed by recombinant retroviruses generated from the cell lines. In the case of lentiviruses, such as HIV-1, HIV-2, SIV, FIV and EIV, the env proteins include gp41 and gp120.

Illustrative examples of env genes which can be employed in the invention include, but are not limited to: MLV envelopes, 10A1 envelope, BAEV, FeLV-B, RD114, SSAV, Ebola, Sendai, FPV (Fowl plague virus), and influenza virus envelopes. Similarly, genes encoding envelopes from RNA viruses (e.g., RNA virus families of Picornaviridae, Calciviridae, Astroviridae, Togaviridae, Flaviviridae, Coronaviridae, Paramyxoviridae, Rhabdoviridae, Filoviridae, Orthomyxoviridae, Bunyaviridae, Arenaviridae, Reoviridae, Birnaviridae, Retroviridae) as well as from the DNA viruses (families of Hepadnaviridae, Circoviridae, Parvoviridae, Papovaviridae, Adenoviridae, Herpesviridae, Poxviridae, and Iridoviridae) may be utilized. Representative examples include, FeLV, VEE, HFVW, WDSV, SFV, Rabies, ALV, BIV, BLV, EBV, CAEV, SNV, ChTLV, STLV, MPMV, SMRV, RAV, FuSV, MH2, AEV, AMV, CT10, and EIAV.

In other embodiments, env proteins suitable for use in particular embodiments include, but are not limited to any of the following viruses: Influenza A such as H1N1, H1N2, H3N2 and H5N1 (bird flu), Influenza B, Influenza C virus, Hepatitis A virus, Hepatitis B virus, Hepatitis C virus, Hepatitis D virus, Hepatitis E virus, Rotavirus, any virus of the Norwalk virus group, enteric adenoviruses, parvovirus, Dengue fever virus, Monkey pox, Mononegavirales, Lyssavirus such as rabies virus, Lagos bat virus, Mokola virus, Duvenhage virus, European bat virus 1 & 2 and Australian bat virus, Ephemerovirus, Vesiculovirus, Vesicular Stomatitis Virus (VSV), Herpesviruses such as Herpes simplex virus types 1 and 2, varicella zoster, cytomegalovirus, Epstein-Bar virus (EBV), human herpesviruses (HHV), human herpesvirus type 6 and 8, Human immunodeficiency virus (HIV), papilloma virus, murine gammaherpesvirus, Arenaviruses such as Argentine hemorrhagic fever virus, Bolivian hemorrhagic fever virus, Sabia-associated hemorrhagic fever virus, Venezuelan hemorrhagic fever virus, Lassa fever virus, Machupo virus, Lymphocytic choriomeningitis virus (LCMV), Bunyaviridiae such as Crimean-Congo hemorrhagic fever virus, Hantavirus, hemorrhagic fever with renal syndrome causing virus, Rift Valley fever virus, Filoviridae (filovirus) including Ebola hemorrhagic fever and Marburg hemorrhagic fever, Flaviviridae including Kaysanur Forest disease virus, Omsk hemorrhagic fever virus, Tick-borne encephalitis causing virus and Paramyxoviridae such as Hendra virus and Nipah virus, variola major and variola minor (smallpox), alphaviruses such as Venezuelan equine encephalitis virus, eastern equine encephalitis virus, western equine encephalitis virus, SARS-associated coronavirus (SARS-COV), West Nile virus, any encephalitis causing virus.

In preferred embodiments, the env gene encodes a VSV-G envelope glycoprotein.

In certain embodiments, the one or more nucleic acids comprise a mixture of one or more plasmid DNAs, wherein the one or more plasmid DNAs comprise, a packaging plasmid, a helper plasmid, and a transfer plasmid encoding an adeno-associated viral (AAV) vector.

In some preferred embodiments, pDNA/PEI complexes contemplated herein comprise a transfer plasmid encoding a lentiviral vector comprising a modified left (5′) lentiviral LTR comprising a heterologous promoter, a Psi packaging sequence (Ψ+), a cPPT/FLAP, an RRE, a promoter operably linked to a polynucleotide encoding a therapeutic transgene, and a modified SIN (3′) lentiviral LTR; a plasmid encoding a lentiviral gag/pol, a plasmid encoding rev, and a plasmid encoding an env gene, preferably a VSV-G envelope glycoprotein.

In certain embodiments, the pDNA in a final suspension of the plurality of stable polycationic/nucleic acid nanoparticles has a concentration in a range of about 50 to about 400 μg/mL, including about 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, and 400 μg/mL.

In certain embodiments, the first buffer comprises one or more components selected from a non-buffering salt, a monovalent buffering salt, a bivalent buffering salt, a trivalent buffering salt, a buffering salt having a higher valency, and combinations thereof. In particular embodiments, the non-buffering salts are selected from a group comprised of sodium chloride, potassium chloride, and combinations thereof; the buffering salts with varying valency are selected from a group comprised of phosphate, citrate, EDTA, pyrophosphate, ATP, tripolyphosphate, hexametaphosphate, bis-Tris, MES monohydrate, Tris base, HEPES, magnesium, calcium, aluminum, Iron (II), Iron (III), and combination thereof.

In certain embodiments, the first buffer has a pH from about 2.0 to about 9.0, including about 2.0, 2.5, 3.0, 3.5, 4.0, 4.5, 5.0, 5.5, 6.0, 6.5, 7.0, 7.5, 8.0, 8.5 and 9.0. In particular embodiments, the first buffer has a pH from about 7.0 to about 7.4, including about 7.0, 7.1, 7.2, 7.3, and 7.4.

In certain embodiments, the second buffer comprises one or more buffering salts selected from a non-buffering salt, a monovalent buffering salt, a bivalent buffering salt, a trivalent buffering salt, a buffering salt having a higher valency, and combinations thereof. In particular embodiments, the non-buffering salts are selected from a group comprised of sodium chloride, potassium chloride, and combinations thereof; the buffering salts with varying valency are selected from a group comprised of phosphate, citrate, EDTA, pyrophosphate, ATP, tripolyphosphate, hexametaphosphate, bis-Tris, MES monohydrate, Tris base, HEPES, magnesium, calcium, aluminum, Iron (II), Iron (III), and combination thereof.

In certain embodiments, the second buffer has a pH from about 2.0 to about 9.0, including about 2.0, 2.5, 3.0, 3.5, 4.0, 4.5, 5.0, 5.5, 6.0, 6.5, 7.0, 7.5, 8.0, 8.5 and 9.0. In certain embodiments, the second buffer comprises an acidic solution.

In certain embodiments, the third buffer comprises an acidic pH buffer having a pH from about 2.0 to about 9.0, including about 2.0, 2.5, 3.0, 3.5, 4.0, 4.5, 5.0, 5.5, 6.0, 6.5, 7.0, 7.5, 8.0, 8.5 and 9.0. In particular embodiments, the acidic pH buffer comprises HCl. In more particular embodiments, the acidic pH buffer comprises between about 0.25 mmol/L to 20 mmol/L of protons, including about 0.25, 0.50, 1, 2, 3, 4, 5, 6, 7, 8, 9, 10, 11, 12, 13, 14, 15, 16, 17, 18, 19, and 20 mmol/L

In certain embodiments, the third buffer comprises one or more sugars. In particular. embodiments, the one or more sugars comprise trehalose. In more particular embodiments, the one or more sugars comprise between about 2.5% to about 30% w/w of trehalose, including about 2.5, 3, 4, 5, 6, 7, 8, 9, 10, 11, 12, 13, 14, 15, 16, 17, 18, 19, 20, 21, 22, 23, 24, 25, 26, 27, 28, 29, and 30% w/w of trehalose.

In certain embodiments, the polycationic polymer is selected from polyethylenimine (PEI), chitosan, PAMAM dendrimers, protamine, poly(arginine), poly(lysine), poly(beta-aminoesters), cationic peptides and derivatives thereof. In particular embodiments, the polycation comprises PEI. In more particular embodiments, the PEI comprises linear PEI or modified linear PEI.

In certain embodiments, the method further comprises incubating the plurality of polycationic/nucleic acid complexes for a period of time having a range from about 0.5 minutes to about 90 minutes, including about 0.5, 1, 2, 3, 4, 5, 6, 7, 8, 9, 10, 15, 20, 25, 30, 35, 40, 45, 50, 55, 60, 65, 70, 75, 80, 85, and 90 minutes. In particular embodiments, the method comprises incubating the plurality of polycationic/nucleic acid complexes at about room temperature (22±4° C.).

In certain embodiments, the defined particle size is between about 100 nm to about 1000 nm, including about 100, 200, 300, 400, 500, 600, 700, 800, 900, and 1000 nm, and any integers in between. In more certain embodiments, the defined particle size is between about 400 nm to about 650 nm, including about 400, 410, 420, 430, 440, 450, 460, 470, 480, 490, 500, 510, 520, 530, 540, 550, 560, 570, 580, 590, 600, 610, 620, 630, 640, and 650 nm, and any integers in between. In certain embodiments, a change in particle size is monitored by a turbidity of the plurality of polycationic/nucleic acid complexes.

In certain embodiments, the first buffer further comprises a cryoprotectant, the second buffer further comprises a cryoprotectant, the third buffer further comprises a cryoprotectant, or combinations thereof. In particular embodiments, the method further comprises lyophilizing or freezing the plurality of polycationic/nucleic acid nanoparticles at about −80° C. to form a plurality of lyophilized or frozen polycationic/nucleic acid nanoparticles. In more particular embodiments, the method further comprises storing the plurality of lyophilized or frozen polycationic/nucleic acid nanoparticles for a period of time. In certain embodiments, the period of time has a range of up to about four months. In certain embodiments, the method further comprises thawing the plurality of lyophilized or frozen polycationic/nucleic acid nanoparticles to form a plurality of thawed polycationic/nucleic acid nanoparticles. In certain embodiments, the plurality of thawed polycationic/nucleic acid nanoparticles are stable for a period of time up to about 4 hours. In certain embodiments, the method further comprises disposing the plurality of thawed polycationic/nucleic acid nanoparticles in a bioreactor to produce one or more transfected cells.

In other embodiments, the presently disclosed subject matter provides a method for preparing a lentiviral vector or adeno-associated viral vector, the method comprising contacting one or more cells with a polycationic/nucleic acid nanoparticle prepared by the method described hereinabove.

In certain embodiments, the one or more cells comprise HEK293 cells or a derivative thereof. In particular embodiments, the one or more cells comprise a cell selected from HEK293S cells, HEK293T cells, HEK293F cells, HEK293FT cells, HEK293FTM cells, HEK293SG cells, HEK293SGGD cells, HEK293H cells, HEK293E cells, HEK293MSR cells, and HEK293A cells. In more particular embodiments, the one or more cells comprise HEK293T cells. In even more particular embodiments, the one or more cells comprise HEK293T cells adapted for suspension culture.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

The “subject” treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein. The term “subject” also refers to an organism, tissue, cell, or collection of cells from a subject.

In general, the “effective amount” of an active agent or drug delivery device refers to the amount necessary to elicit the desired biological response. As will be appreciated by those of ordinary skill in this art, the effective amount of an agent or device may vary depending on such factors as the desired biological endpoint, the agent to be delivered, the makeup of the pharmaceutical composition, the target tissue, and the like.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount, or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### Example 1

### Kinetically Controlled Methods to Prepare Concentrated Shelf-Stable Nucleic Acid Transfection Vehicles

### 1.1 Overview

The presently disclosed subject matter, in part, provides tool kits and protocols for preparing transfection vehicles, including viral vectors and other biological products, for large-scale in vitro transfection purposes. In particular, the presently disclosed subject matter provides procedures for the simplification of the generation of transfection complexes having a size/dimension greater than 400 nm.

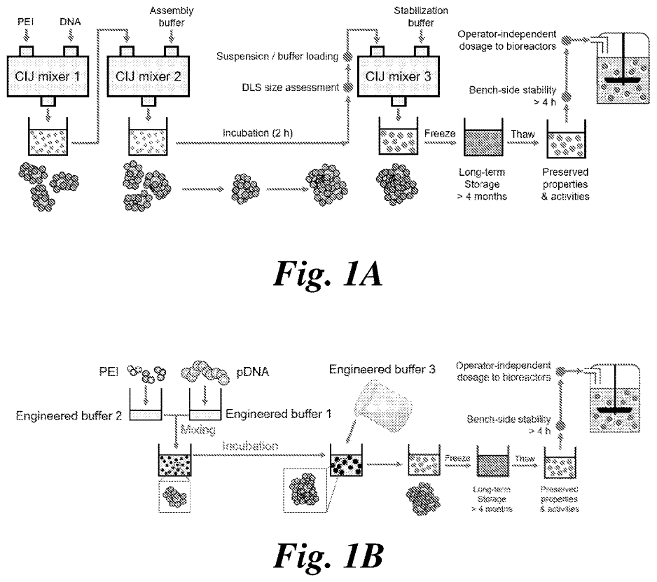

Referring now to FIG. 1A and FIG. 1B are schematics of methods previously disclosed in the '980 application and the '981 application (FIG. 1A) and the presently disclosed methods (FIG. 1B). Further comparisons between the previously disclosed methods and the presently disclosed methods are provided in Table 1.

TABLE 1
Key features between the presently disclosed and previously published methods

| | Previous methods (2021 Nano | |
|---|---|---|
| | Letters) and follow up | Presently disclosed methods |
| Apparatus | CIJ mixers at sufficiently high | Common mixing devices, |
| requirements | flow rates to achieve turbulent | batch mixing or pumps with |
| | mixing | moderate to low flow rates |
| Maximum validated | 50 μg DNA/mL | 50-400 μg/mL |
| DNA concentration | | |
| Validated | 5000 mL | 80 mL at 180 μg/mL of |
| production volume | | pDNA (equivalent to around |
| | | 290 mL at 50 μg/mL pDNA) |
| | | With no conceivable |
| | | difficulty in further scaling up |
| Time needed for | 3 flash mixing steps | Two simple mixing steps: |
| particle assembly | *To produce 1000 mL: >4 h | 20-40 min |
| | not including preparation of | |
| | solutions | |
| Low batch-to-batch | Yes | Yes |
| variability | | |
| Coverage of optimal | Yes | Yes |
| size range (e.g., 400 | | |
| nm to 600 nm) | | |
| Greater than 4 h | Yes | Yes |
| bench-side stability | | |
| at room temperature | | |
| Shelf stability under | Yes | Yes |
| cold storage | | |

### 1.2 Summary

The key components of this Example are a set of buffers that can be used to dilute PEIpro® and a DNA cocktail to improve the procedures to generate transfection complexes. The general procedures can be described as follows: Use DNA dilution buffer to dilute a DNA cocktail. Use PEI dilution buffer to dilute the supplied PEIpro® solution. With a mixing mechanism (stirred containers, liquid handlers, orbital shakers, and the like), add buffered PEI into buffered DNA. Without the need of strict timing (anywhere from 5 to 25 min), add 10% vol % stabilization buffer into the mixture. To improve robustness, this process can be monitored by plate-based absorbance assay. The pDNA/PEI complexes are immediately stable (both on benchtop and in cold storage).

Due to the size control mechanisms, it is believed that this process is independent of exact mixing mechanisms; operator-independent; independent of mixing scales; compatible with very high complex concentrations. The only requirement on the reagents is that the stock DNA solutions must be dissolved in pure water, rather than a pH buffer with salt.

The DNA dilution buffer contains certain concentrations of salts that are monovalent, and/or bivalent, and/or trivalent, and/or with higher valency, at a pH ranging from 2.0 to 9.0, with addition of cryoprotectants and other excipients.

PEI dilution buffer contains certain concentrations of salts that are monovalent, and/or bivalent, and/or trivalent, and/or with higher valency, at a pH ranging from 2.0 to 9.0, with addition of cryoprotectants and other excipients.

Stabilization buffer contains certain concentrations of HCl at a pH ranging from 2.0 to 9.0 with addition of cryoprotectants and other excipients.

### Example 2

### Kinetically Controlled Methods to Prepare Concentrated Shelf-Stable Nucleic Acid Transfection Vehicles

### 2.1 Experimental Reagents, Materials and Methods

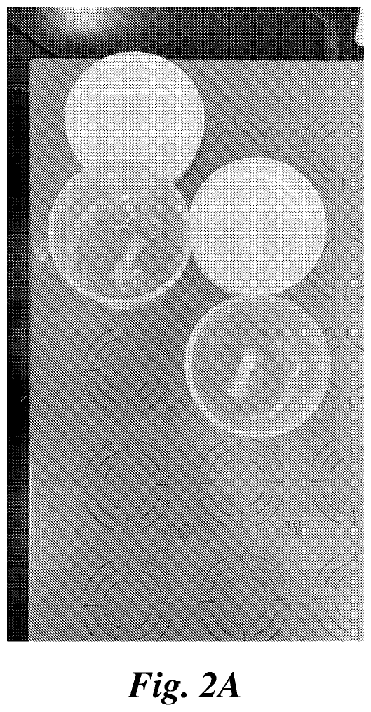

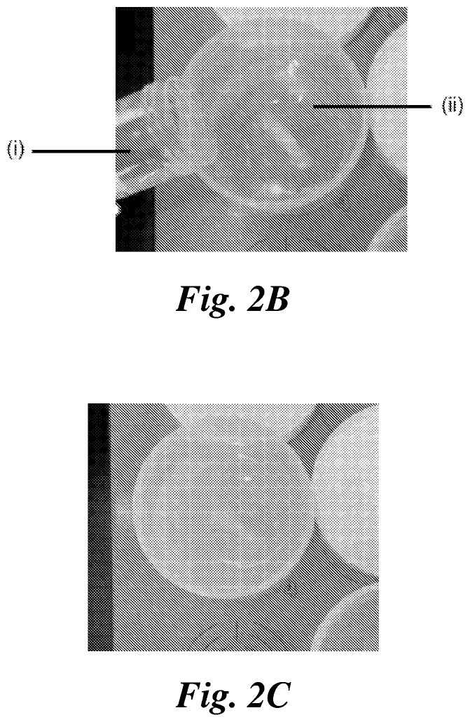

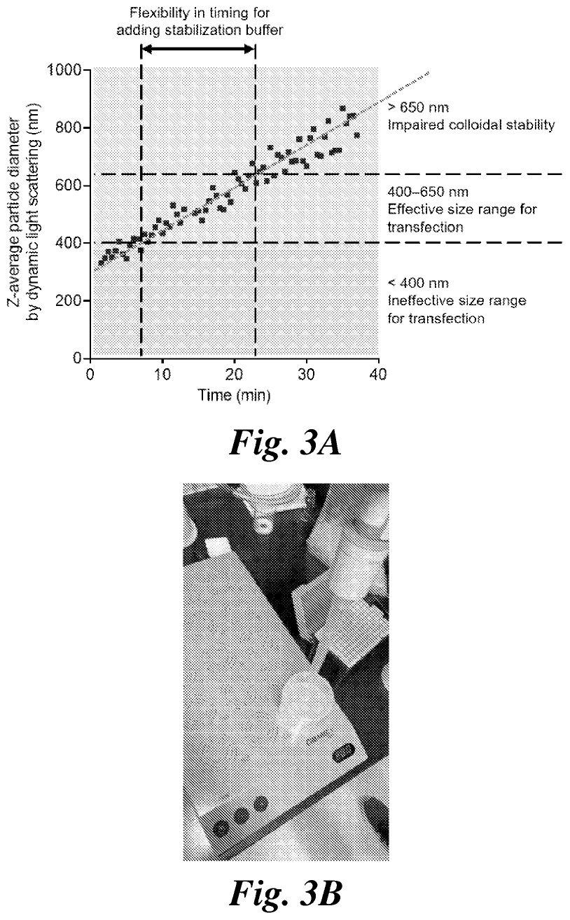

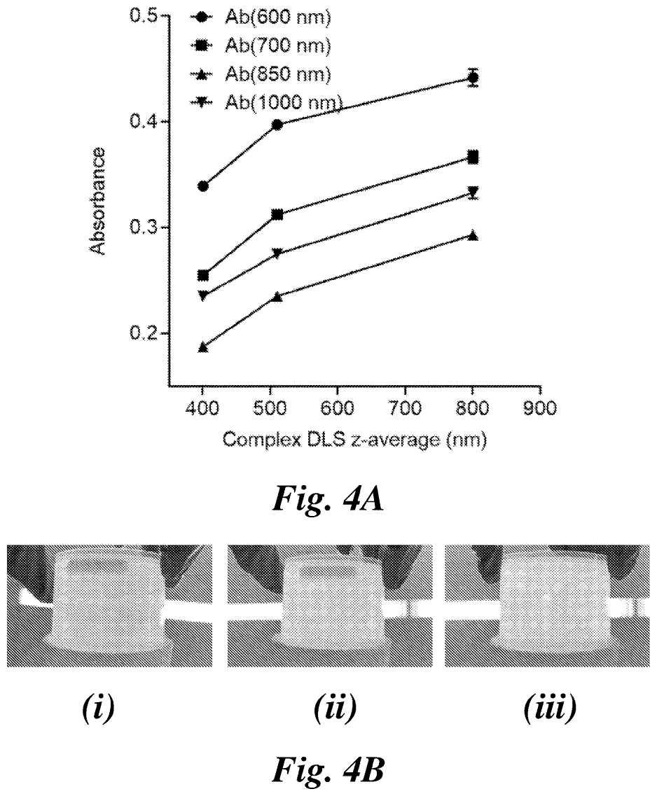

Luciferase DNA (gWiz-Luc, 6732 kb, Aldevron LLC) or tdTomato DNA (custom synthesized through Aldevron LLC) was dissolved in the DNA dilution buffer (Engineered buffer 1, or the “first buffer” as described in claim 1, containing 0.5×PBS and 2.5 mM sodium citrate in 19% (w/w) trehalose solution) at a concentration of 440 μg/mL. The polycation, i.e., poly(ethyleneimine) (PEIpro® from Polyplus, Inc.) was dissolved in the PEI dilution buffer (Engineered buffer 2, or the “second buffer” as described in claim 1, 19% (w/w) trehalose solution) at a concentration of 580.8 μg/mL that was equivalent to a nitrogen-to-phosphate ratio of 5.5. A typical formulation process to obtain particles with defined sizes is described FIG. 1B, involving three steps: Step 1, or complexation; Step 2, or incubation; and Step 3, or stabilization. The mixing for Step 1 and Step 3 can be implemented by bulk mixing (by a pipette, by pouring with beakers, as shown in FIG. 2A-FIG. 2C) or pump-mediated mixing (through a confined impinging jet or CIJ mixer). When the size of the growing particles reached the target size, a stabilization buffer (Engineered buffer 3, or the “third buffer” as described in claim 1) of 20 mM HCl in 9.5% (w/w) trehalose solution was added to the growing particle suspension at a 10% volumetric ratio, generating stabilized particles with a defined size.

For transfection tests, HEK293T cells were seeded at 100,000 cells/well in a 24-well plate, 1 day prior to transfection. Stabilized particles at a DNA concentration of 200 μg/mL, containing 10% luciferase plasmid with 90% non-coding plasmid, or 100% tdTomato plasmid were diluted by Opti-MEM medium. The stabilized particles were dosed into the cells at 1 μg DNA per well, and incubated for 4 h, followed by medium exchange with fresh medium and cultured for an additional 44 h. Cells were then lysed with 200 μL reporter lysis buffer (Cat. #E4030, Promega Co.), and the lysates underwent a freeze-thaw cycle at −80° C. For luciferase measurements, 20 μL of each sample was mixed with 100 μL luciferase substrate (Cat. #E1483, Promega Co.) and the luminescence signal was measured. For tdTomato fluorescence assessment, Attune™ NxT Flow Cytometer (Thermo Fisher Scientific Inc.) was used. Cells were stained with LIVE/DEAD™ Fixable Aqua Dead Cell Stain Kit (Thermo Fisher Scientific Inc.) for viability assessment before being analyzed by the flow cytometer.

HEK293F cells were seeded at 1,000,000 cells/well in a 12-well plate, on the day of transfection. Stabilized particles at a DNA concentration of 200 μg/mL, containing 10% luciferase plasmid with 90% non-coding plasmid, or 100% tdTomato plasmid were added directly into the cell suspension at 1 μg DNA per well. Cells were incubated for 48 h until further analysis. For luciferase assessment, cells were collected from the plates and lysed with 500 μL reporter lysis buffer; and the luminescence assay was conducted using the same protocol as described above. The tdTomato fluorescence assessment using flow cytometry was conducted using the same protocol as described above.

### 2.2 Results

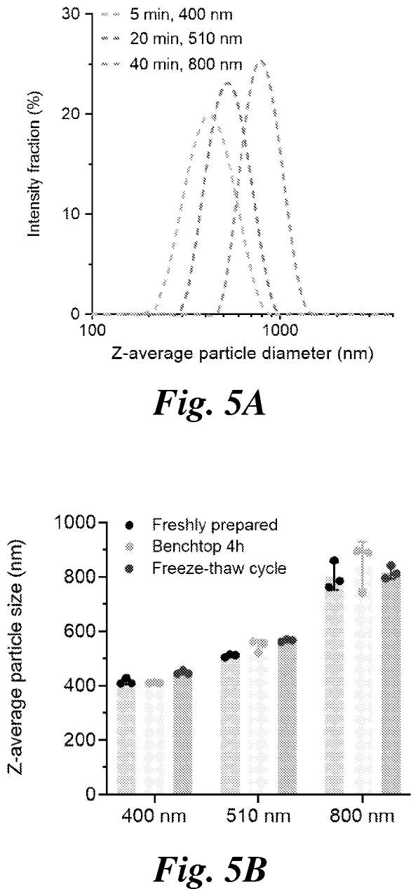

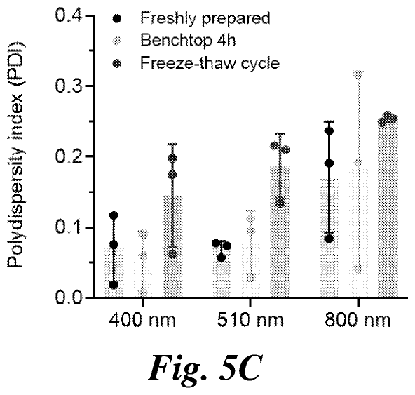

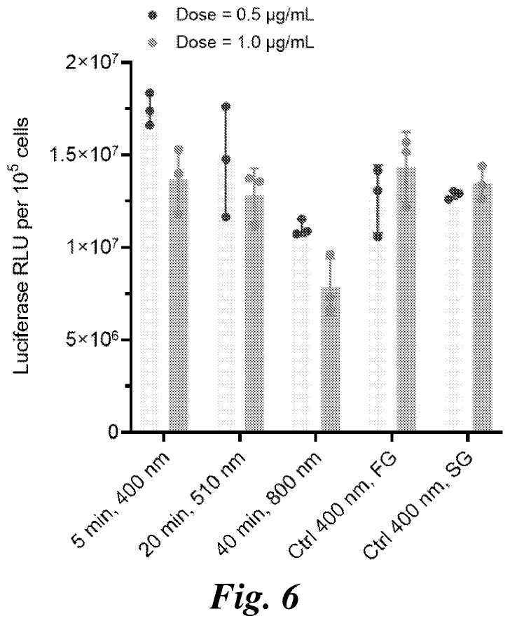

The production method was carefully designed to streamline the assembly process of pDNA/PEI particles to generate high-concentration, shelf-stable particles. Upon mixing, the pDNA/PEI particles initiate an instantaneous assembly with a controlled rate of size growth. The size growth is mediated by the salt ions present in the solution, particularly multivalent ions, deprotonating PEI to reduce repulsion by increasing pH, providing charge shielding, and inducing the particle association. The optimal Z-average size of the particles can be obtained when stabilization occurs at a desired time point throughout the assembly process. The time window for incubation (Step 2) to achieve the highest transfection efficiency ranged from 20 to 40 min., which can be further tuned by modifying the ionic strength of the first and the second buffer if desired. By balancing the forces between the repulsion among positively charged particles and the attraction induced by charge screening, the particles showed substantially improved stability both on bench for hours and resistance to freeze-thaw cycle (FIG. 5).

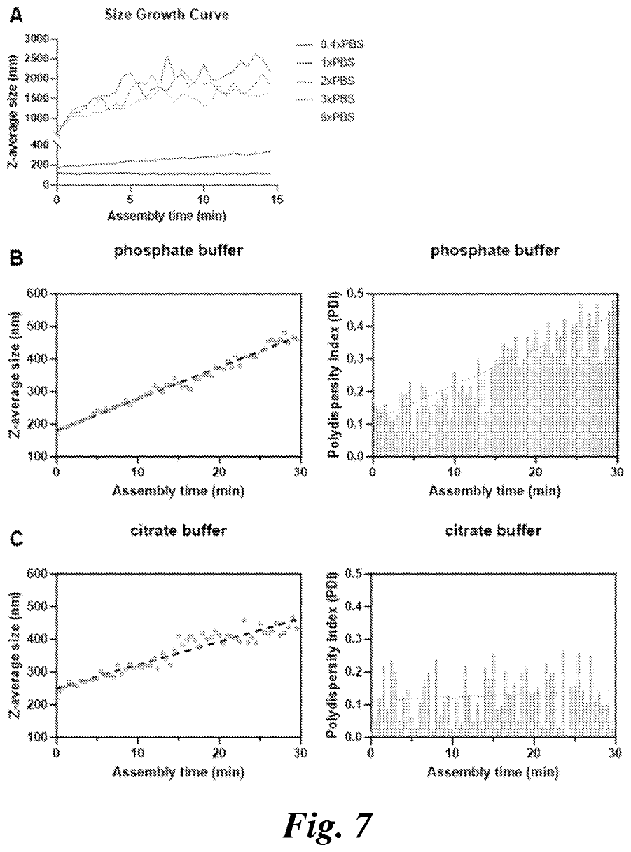

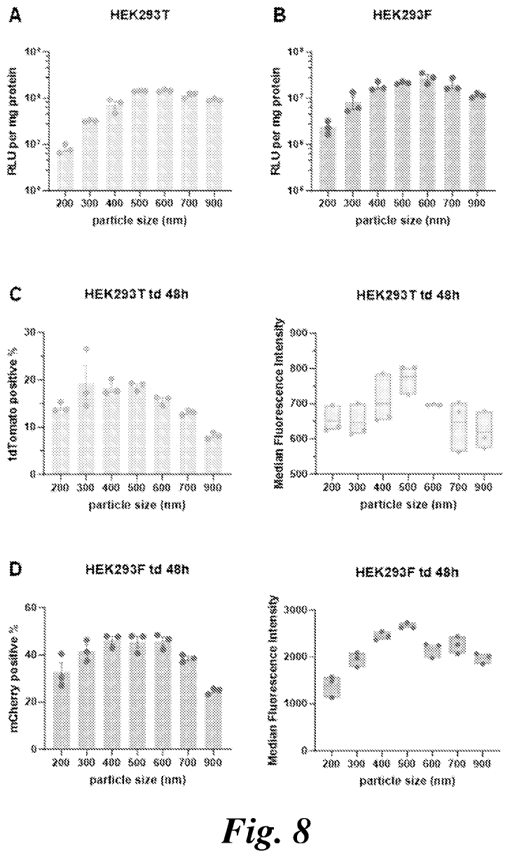

Notably, with the presence of multivalent ions (specifically citrate in this case), the DNA concentration in the final particle product achieved up to 200 μg/mL, which is 10-fold higher compared to conventional complexation methods and could not be obtained with PBS buffer alone (FIG. 7A). When increasing the concentration of pDNA in assembly mediated by PBS, the complexes exhibited heterogenous assembly indicated by high polydispersity (>0.4) at size above 400 nm (FIG. 7B), due to the high concentration of both materials, rendering the phosphate buffer ineffective in inducing uniform assembly. However, the high concentration of DNA did not compromise the uniformity and stability of the particles, indicated by PDI less than 0.3 at each point, when citrate ion is used for assembly (FIG. 7C). This is a remarkable improvement for transfection in industrial scale setting, where bioreactors are typically in litter scale. Therefore, the dosing volume of the particles to the bioreactor can also be reduced to 10-fold as a result of increased particle concentration.

The transfection results from luciferase measurements as an indicator of total protein expression level exhibited a size-dependent efficiency for both HEK293T cells and HEK293F cells (FIG. 8A, FIG. 8B). The optimal transfection window lies between 400 nm to 600 nm regarding particle size, indicating that larger size particles carry more DNA copies thus expressing more after transfecting the cells without overwhelming them. Our previous study also demonstrated that particles with larger sizes exhibited higher cellular uptake and increased endosomal escape level (10.1021/acsnano.9b03334) which positively affect the transfection efficiency. These findings were further confirmed by flow cytometry measuring the percentage of tdTomato-positive cells, and the average expression at single-cell level indicated by the median fluorescence intensity (FIG. 8C, FIG. 8D). The highest transfection efficiency was achieved by a particle size between 400 nm to 600 nm, indicating that the size directly influences the transfection efficiency for this series of particles generated by this method.

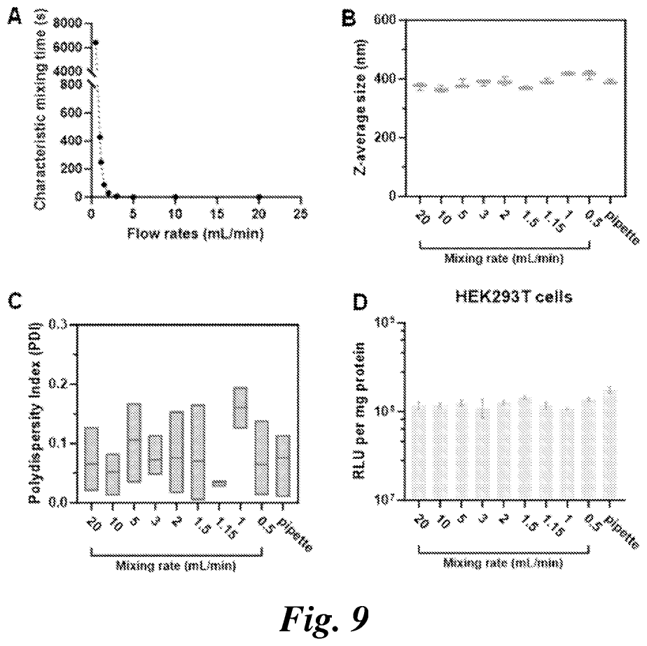

To examine the sensitivity of the particle manufacturing process to the exact mixing modality and/or mixing rate used for Step 1 and Step 3, a pump-controlled CIJ mixer was used with the flow rate varied to achieve a series of characteristic mixing time. Pipette mixing (with a volume of 1 mL mixing with 1 mL) was also used as a control. Our previous publication (10.1021/acsnano.9b03334) demonstrated that the mixing rate and play a crucial role in producing homogeneous DNA/PEI nanoparticles. Typically, slower mixing results in more heterogeneous complexes of DNA and PEI due to the inadequate degree of mixing, leading to a slower diffusion of the two solutions. In contrast, with the buffers and methods described in FIG. 1B, we confirmed that employing different mixing methods, regardless of bulk mixing (pipetting) or pump-controlled CIJ mixing at varying flow rates which covered a wide range of characteristic mixing times (FIG. 9A), yield target 400-nm DNA/PEI particles with consistent size and homogeneity. Specifically, irrespective of the characteristic mixing time, the Z-average size of these DNA/PEI complexes remained consistent when particles were stabilized at the same time point (approximately 15 min) (FIG. 9B). They also exhibited good uniformity indicated by a polydispersity index (PDI) that was consistently lower than 0.2 after stabilization (FIG. 9C). This observation demonstrated the presence of kinetic control in the system, eliminating the need for developing mixing devices to achieve better particle uniformity. Furthermore, we transfected the HEK293T cell line using the particles formulated by different characteristic mixing times. The consistent transfection efficiencies observed across this series of particles further validated their coherent quality (FIG. 9D).

### 2.3 Discussion

Altogether, these data demonstrated that the kinetically controlled method for generating DNA/PEI complexes described in this invention can deliver shelf-stable particles with defined sizes and consistent quality, and the transfection efficiency can be optimized regarding particle size simply by controlling incubation time. This kinetically controlled method not only provides an optimal size window for improved transfection efficiency, but also simplifies and streamlines the process to produce shelf-stable particles. Notably, it addresses the problem of low DNA concentration in the DNA/PEI particles simultaneously which burdens the dosing process in industrial production of viral vectors. This method can be easily implemented across scales, with insensitivity to the exact mixing quality attainable during the production process and is thus generalizable among various viral vector production processes mediated by PEI-based transfection.

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art.
International PCT Patent Application Publication No. WO2022177980 for Methods for Preparation of Shelf-Stable Plasmid DNA/Polycation Particles with Defined Sizes for Cell Transfection, to Mao et al., published Aug. 25, 2022.
International PCT Patent Application Publication No. WO2022177981 for Composition of Shelf-Stable Plasmid DNA/Polyethyleneimine Particles With Defined Sizes For Viral Vector Production and Methods for Preparation, to Mao et al, published Aug. 25, 2022.
Hu Y, He Z, Hao Y, Gong L, Pang M, Howard G P, Ahn H H, Brummet M, Chen K, Liu H W, Ke X, Zhu J, Anderson C F, Cui H, Ullman C G, Carrington C A, Pomper M G, Seo J H, Mittal R, Minn I, Mao H Q. Kinetic Control in Assembly of Plasmid DNA/Polycation Complex Nanoparticles. ACS Nano. 2019 Sep. 24; 13(9):10161-10178.
Hu Y, Zhu Y, Sutherland N D, Wilson D R, Pang M, Liu E, Staub J R, Berlinicke C A, Zack D J, Green J J, Reddy S K, Mao H Q. Size-Controlled and Shelf-Stable DNA Particles for Production of Lentiviral Vectors. Nano Lett. 21(13): 5697-570 (2021).

Although the foregoing subject matter has been described in some detail by way of illustration and example for purposes of clarity of understanding, it will be understood by those skilled in the art that certain changes and modifications can be practiced within the scope of the appended claims.

That which is claimed:

1. A method for preparing a plurality of stable polycationic/nucleic acid complex nanoparticles, the method comprising:
(a) providing one or more nucleic acids in a first buffer in a total nucleic acid concentration ranging from 0.1 to 0.8 mg/mL;
(b) providing a polycationic polymer in a second buffer, wherein the first buffer and the second buffer can be the same or different;
(c) mixing the polycationic polymer with the nucleic acid and incubating mixture for a period of time to form a plurality of polycationic/nucleic acid complexes; and
(d) mixing a third buffer with the plurality of polycationic/nucleic complexes to form a plurality of stable polycationic/nucleic acid nanoparticles having a defined particle size.

2. The method of claim 1, wherein the one or more nucleic acids are selected from an antisense oligonucleotide, cDNA, genomic DNA, guide RNA, plasmid DNA (pDNA), vector DNA, mRNA, miRNA, piRNA, shRNA, siRNA, and a mixture of different species of plasmid DNA.

3. (canceled)

4. The method of claim 2, wherein the one or more nucleic acids comprise: (a) a mixture of one or more plasmid DNAs, wherein the one or more plasmid DNAs comprise a transfer plasmid encoding a lentiviral vector and plasmid DNAs encoding a gag protein, a pol protein, a rev protein, and an env protein; or (b) a mixture of one or more plasmid DNAs, wherein the one or more plasmid DNAs comprise, a packaging plasmid, a helper plasmid, and a transfer plasmid encoding an adeno-associated viral (AAV) vector.

5. The method of claim 4, wherein the lentiviral vector comprises a modified left (5′) lentiviral LTR comprising a heterologous promoter, a Psi packaging sequence (Ψ+), a cPPT/FLAP, an RRE, a promoter operably linked to a polynucleotide encoding a therapeutic transgene, and a modified SIN (3′) lentiviral LTR.

6. The method of claim 4, wherein the env protein comprises a VSV-g envelope glycoprotein.

7. (canceled)

8. The method of claim 2, wherein the pDNA in a final suspension of the plurality of stable polycationic/nucleic acid nanoparticles has a concentration in a range of about 50 to 400 μg/mL.

9. The method of claim 1, wherein the first buffer and/or the second buffer independently comprise one or more components selected from a non-buffering salt, a monovalent buffering salt, a bivalent buffering salt, a trivalent buffering salt, a buffering salt having a higher valency, and combinations thereof.

10. The method of claim 9, wherein the non-buffering salts are selected from a group comprised of sodium chloride, potassium chloride, and combinations thereof; the buffering salts with varying valency are selected from a group comprised of phosphate, citrate, EDTA, pyrophosphate, ATP, tripolyphosphate, hexametaphosphate, bis-Tris, MES monohydrate, Tris base, HEPES, magnesium, calcium, aluminum, Iron (II), Iron (III), and combinations thereof.

11. The method of claim 9, wherein the first buffer has a pH range selected from about 2.0 to about 9.0 and about 7.0 to about 7.4.

12-14. (canceled)

15. The method of claim 1, wherein the second buffer comprises an acidic solution.

16. The method of claim 1, wherein the third buffer comprises an acidic pH buffer having a pH from about 2.0 to about 9.0.

17. The method of claim 16, wherein the acidic pH buffer comprises HCl.

18. The method of claim 17, wherein the acidic pH buffer comprises between about 0.25 mmol/L to 20 mmol/L of protons.

19. The method of claim 1, wherein the third buffer comprises one or more sugars.

20. The method of claim 19, wherein the one or more sugars comprise trehalose.

21. The method of claim 20, wherein the one or more sugars comprise between about 2.5% to about 30% w/w of trehalose.

22. The method of claim 1, wherein the polycationic polymer is selected from polyethylenimine (PEI), chitosan, PAMAM dendrimers, protamine, poly(arginine), poly(lysine), poly(beta-aminoesters), cationic peptides and derivatives thereof.

23. (canceled)

24. The method of claim 22, wherein the PEI comprises linear PEI or modified linear PEI.

25. The method of claim 1, comprising incubating the plurality of polycationic/nucleic acid complexes for a period of time having a range from about 0.5 minutes to about 90 minutes at about room temperature (22±4° C.).

26. (canceled)

27. The method of claim 1, wherein the defined particle size is has a range selected from between about 100 nm to about 1000 nm and about 400 nm to about 650 nm.

28. (canceled)

29. The method of claim 1, wherein the first buffer further comprises a cryoprotectant, the second buffer further comprises a cryoprotectant, the third buffer further comprises a cryoprotectant, or combinations thereof.

30. The method of claim 29, further comprising one or more of:
(a) lyophilizing or freezing the plurality of polycationic/nucleic acid nanoparticles at about −80° C. to form a plurality of lyophilized or frozen polycationic/nucleic acid nanoparticles;
(b) storing the plurality of lyophilized or frozen polycationic/nucleic acid nanoparticles for a period of time, wherein the period of time has a range of up to about four months; and/or
(c) thawing the plurality of lyophilized or frozen polycationic/nucleic acid nanoparticles to form a plurality of thawed polycationic/nucleic acid nanoparticles, wherein the plurality of thawed polycationic/nucleic acid nanoparticles are stable for a period of time up to about 4 hours.

31-34. (canceled)

35. The method of claim 30, further comprising disposing the plurality of thawed polycationic/nucleic acid nanoparticles in a bioreactor to produce one or more transfected cells.

36. A method for preparing a lentiviral vector or adeno-associated viral vector, the method comprising contacting one or more cells with a polycationic/nucleic acid nanoparticle prepared by the method of claim 1.

37. The method of claim 36, wherein the one or more cells comprise HEK293 cells or a derivative thereof.

38. The method of claim 37, wherein the one or more cells comprise a cell selected from HEK293S cells, HEK293T cells, HEK293F cells, HEK293FT cells, HEK293FTM cells, HEK293SG cells, HEK293SGGD cells, HEK293H cells, HEK293E cells, HEK293MSR cells, and HEK293A cells.

39. (canceled)

40. The method of claim 38, wherein the one or more cells comprise HEK293T cells adapted for suspension culture.
